(19) United States
(12) Patent Application Publication
(10) Pub. No.: US 2001/0049833 A1
(43) Pub. Date: Dec. 6, 2001

(54) DNA constructs comprising protease encoding sequences or inhibitors thereof

(76) Inventors: Andrew James Greenland, Bracknell (GB); Didier Rene Philippe Thomas, Bracknell (GB); Ian Jepson, Bracknell (GB)

Correspondence Address: HALE AND DORR, LLP, 60 STATE STREET, BOSTON, MA, 02109

(21) Appl. No.: 09/782,982

(22) Filed: Feb. 14, 2001

Related U.S. Application Data
(63) Continuation of application No. PCT/GB99/02699, filed on Aug. 16, 1999.

(30) Foreign Application Priority Data
Aug. 17, 1998 (GB) 9817909.6

(51) Int. Cl. A01H 5/00; C12N 15/82; C12N 5/14; C12N 5/16

(52) U.S. Cl. 800/298; 435/320.1; 435/419; 435/325; 800/278

(57) ABSTRACT

Disclosed are isolated DNA constructs including (a) a first DNA sequence including either an inducible promoter sequence responsive to the presence or absence of an exogenous inducer or a developmental gene promoter capable of initiating gene expression in a selected tissue or at a selected stage of development of an organism; (b) a second DNA sequence including a DNA sequence coding for a protease enzyme operably linked and under the control of the promoter sequence specified at (a); whereby the presence or absence of the exogenous inducer or the activation of the developmental gene promoter specified at (a) results in expression of the protease enzyme. These constructs are preferably rendered reversible by the presence of further elements. These constructs can be used in plant or mammalian cells for disruption of cell function, controlling senescence and modifying the metabolism of stored proteins.

The present invention relates to DNA constructs, for use in transformations of plant and mammalian cells. In particular, the present invention relates to a DNA construct which enables cell function to be disrupted and, optionally, for the disruption of cell function to be reversed. The present invention also relates to a DNA construct which enables the process of senescence in plants to be slowed down or inhibited.

A further DNA construct according to the present invention enables the process of protein metabolism, in particular of stored protein, to be modified in order to improve plant performance or yield. Such constructs may be used to delay the germination of seed as required to prevent pre-harvest sprouting.

The DNA constructs of the present invention therefore make use of DNA sequences which are able to inhibit proteins required for cell function, or which control senescence or germination and/or which control protein metabolism, in particular of stored protein. DNA sequences such as those encoding precursor proteins are useful in the present invention.

Proteins having a pre-pro-enzyme or pro-enzyme precursor protein are particularly useful in the present invention. Examples of proteins having such a precursor protein include protease enzymes. One such protease is a cysteine protease. Cysteine proteases (CPs) are members of a large multigene family in plants (Praekelt et al., 1988 ; Goetting-Minesky and Mullin, 1994), animals (Wiederanders et al., 1992) and protozoa (Mallinson et al, 1994). Cysteine proteases are synthesised as an inactive precursor (Praekelt et al., 1988). The pre-pro-enzyme is targeted to the secretory pathway (Marttila et al.,1995) and is post-transcriptionally processed in the vacuoles by proteolytic cleavage of the propeptide fragment to produce the active enzyme (Hara-Nishimura et al., 1993 and 1994).

Plant cysteine proteases participate in different metabolic events of physiological importance. During seed germination and plant senescence they are involved in protein degradation (Jones et al., 1995; Valpuesta et al., 1995; Smart et al., 1995) and play a key role in the mobilisation of stored protein during germination (Boylan and Sussex, 1987). During seed development, cysteine proteases catalyse the post-translational processing of protein precursors into their mature form (Hara-Nishimura et al., 1995). In addition, some are subjected to hormonal regulation either by giberellic acid (Koehler and Ho, 1990; Watanabe et al., 1991) or ethylene (Cervantes et al., 1994; Jones et al., 1995). Others are induced in response to stress like wounding (Linthorst et al., 1993; Lidgett et al., 1995), dehydration (Guerrero et al., 1990), cold (Schaffer and Fischer, 1988) or are implicated in plant-microbe interactions (Goetting-Minesky and Mullin, 1994).

Germination specific cysteine proteases have been characterised for barley (Marttila et al., 1995), rice (Watanabe et al., 1991), maize (Debarros and Larkins, 1994), chick-pea (Cervantes et al., 1994), vetch (Becker et al, 1994) and a cysteine protease has been described for oil seed rape (Comai and Harada, 1989).

Taylor et al., 1995 describe the study in vitro of proteolytic enzymes. A number of proteases are described including members of the cyteine protease family. Recombinant proregions of papain and papaya protease IV, produced in E. coli, can act as differential fast binding inhibitors of the four naturally occuring papaya cysteine proteases. It is taught that evidence suggests that the cleavable “pro” regions in protease precursors function not only to inhibit protease activity but also to assist folding. Recombinant propeptides expressed in E. coli were found to be selective inhibitors of their cognate protease in the nanomolar range, while other-cysteine proteases tested were unaffected by the presence of the propeptide up to 10 micromolar (Volkel et al., 1996). There is no teaching or suggestion, however, of any use for such proteases or any teaching of the DNA constructs of the present invention.

It is desirable to provide methods of controlling functions such as reversible cell disruption, senescence and stored protein metabolism in an organism.

Thus the invention provides a DNA construct comprising:

a) a first DNA sequence comprising either an inducible promoter sequence responsive to the presence or absence of an exogenous inducer or a developmental gene promoter capable of initiating gene expression in a selected tissue or at a selected stage of development of an organism;

b) a second DNA sequence comprising a DNA sequence coding for a protease enzyme operably linked and under the control of the promoter sequence specified at (a);

whereby the presence or absence of the exogenous inducer or the activation of the developmental gene promoter specified at (a) results in expression of said protease enzyme.

The use of exogenous inducers to control promoter activity is well known. Promoters may be stimulated by a variety of factors, for example, environmental conditions, presence of a pest or pathogen or the presence of a chemical. In particular, suitable inducible promoters are induced by an exogenous chemical stimulus such that the exogenous inducer is a chemical. The external chemical stimulus is preferably an agriculturally acceptable chemical, the use of which is compatible with agricultural practice and is not detrimental to plants or mammals.

The inducible promoter most preferably comprises an inducible switch promoter system such as, for example, a two component system such as the alcA/alcR gene switch promoter system described in our published International Publication No. WO 93/21334, the ecdysone switch system as described in our International Publication No. WO 96/37609 or the GST promoter as described in published International Patent Application Nos. WO 90/08826 and WO 93/031294, the teachings of which are incorporated herein by reference. Such promoter systems are herein referred to as “switch promoters”. The switch chemicals used in conjunction with the switch promoters are agriculturally acceptable chemicals making this system particularly useful in the method of the present invention.

Similarly, development specific promoters, are also well known. For example some promoters, such as the malate synthase (MS) promoter (see Graham et al., 1990, Plant Mol Biol. 15, 539-549, Comai et al 1992, Plant Physiol., 98, 53-61), are active during early seedling development in plants. Further examples include promoters of cysteine protease promoters themselves, such as those described in WO 97/35983. Alternatively, plant development promoters from genes in the glyoxysome such as isocitrate lyase, and promoters from genes in the aleurone layer such as α-amylases (Baulcombe et al. (1987) Mol. & Gen Genet. 209, 33-40). Scutellum gene promoters such as that of carboxypeptidase or promoters from germin genes (Lane et al., (1991) J. Biol. Chem. 266, 10461) may also comprise promoters which are active at a particular stage in the development of plants.

Promoters active at a selected phase of development may also be isolated using conventional methods, for example by applying a reverse transcriptase polymerase chain reaction (RT-PCR) strategy to RNA from organisms at the selected development stage, and comparing these using RNA blot analysis with RNA from cells at different development stages. These sequences can then be identified and sequenced conventionally and the promoter sequences determined. For a particular application of the present, as described hereinafter, senescence-induced promoter.

Tissue specific promoters are also known in the art or can be isolated using similar methods. Examples of known tissue specific promoters include anther- and/or tapetum-specific promoter or a pollen-specific promoter. Other such promoters can be isolated in particular by using the techniques described in International Patent Application No. WO 90/08826.

The term “protease enzyme” used herein refers to a naturally occuring protease enzyme or a fragment thereof or a variant of either of these, provided it has protease activity.

The term “variant” as used herein includes experimentally generated variants or members of a family of related naturally-occurring peptides as may be identified by molecular genetic techniques. Such techniques are described for example in U.S. Pat. Nos. 5,605,793, 5,811,238 and 5,830,721, the content of which is incorporated herein by reference. In essence this technique involves expression of the parental gene in a microbial expression system such as Escherichia coli. The particular system selected must be validated and calibrated to ensure that biologically active peptides are expressed, which may be readily achieved using a in vivo bioassay. The gene, or preferably a collection of related genes from different species, may be subject to mutagenic polymerase chain reaction (PCR) as is known in the art. Fragmentation of the products and subsequent repair using PCR leads to a series of chimeric genes reconstructed from parental variants. These chimeras are then expressed in the microbial system which can be screened in the usual way to determine active mutants, which may then be isolated and sequenced. Reiteration of this molecular evolution DNA shuffling cycle may lead to progressive enhancement of the desired gene properties. The advantage of a technique of this nature is that it allows a wide range of different mutations, including multi-mutation block exchanges, to be produced and screened.

Other variants are those which are experimentally generated using for example the molecular evolution techniques. Preferably such variants will have improved activity or function as compared to the native sequences. Suitable improvements may be in relation to the intrinsic specific activity of the protein, or by altering a physical property such as stability.

Other variants may be identified or defined using bioinformatics systems. An example of such a system is the FASTA method of W. R. Pearson and D. J. Lipman PNAS (1988) 85:2444-2488. This method provides a rapid and easy method for comparing protein sequences and detecting levels of similarity and is a standard tool, used by molecular biologists. Such similar sequences may be obtained from natural sources, through molecular evolution or by synthetic methods and comparisons made using this method to arrive at “opt scores” which are indicative of the level of similarity between the proteins.

With these constraints in mind, a skilled person would be able to isolate other members of the family of peptides, for example by designing probes or primers based upon a naturally occurring protease enzyme but modified within the limits of the FASTA opt score range. These probes could then be used to screen libraries such as cDNA or genomic libraries using conventional methods, in order to isolate other enzymes with similar activity. Hybridisation conditions used during these screening exercises are either low or high stringency, preferably high stringency conditions as are routinely used in the art (see for example “Molecular Cloning, A Laboratory Manual” by Sambrook et al, Cold Spring Harbor Laboratory Press, N.Y. ). In general terms, low stringency conditions can be defined as 3×SCC at about ambient temperature to about 65° C., and high stringency conditions as 0.1×SSC at about 65° C. SSC is the name of a buffer of 0.15 M NaCl, 0.015 M trisodium citrate. 3×SSC is three times as strong as 1×SSC and so on.

Once found other family members could also be subject to molecular evolution techniques or DNA shuffling as described herein, in order to improve the properties thereof. All peptides obtained in this way should be regarded as a variant.

In a second aspect, the invention provides plant germplasm comprising a DNA construct as described above.

Expression of protease enzymes in a controllable manner may be used for a variety of purposes. For example, they may be used to disrupt cell function which may find application for example in the production of male sterile plants as described for example in WO 90/08830 Preferably in such an instance, the disruption of cell function is reversible. This can be achieved in the present case at the protein level, by controllably expressing a protein which disrupts the protease enzyme.

Thus, according to a third aspect of the present invention there is provided a DNA construct comprising:

a) a first DNA sequence comprising either an inducible promoter sequence responsive to the presence or absence of an exogenous inducer or a developmental gene promoter capable of initiating gene expression in a selected tissue or at a selected stage of development of an organism;

b) a second DNA sequence comprising a DNA sequence coding for a protease enzyme capable of disrupting cell function operably linked and under the control of the promoter sequence specified at (a);

c) a third DNA sequence comprising a promoter sequence responsive to the presence or absence of an exogenous inducer or a developmental gene promoter capable of initiating gene expression in a selected tissue or at a selected stage of development of an organism; and

d) a fourth DNA sequence comprising a DNA sequence, the product of which is capable of inhibiting the protein specified in (b) operably linked and under the control of the promoter sequence specified at (c),

whereby the presence or absence of the exogenous inducer or the expression of the developmental gene promoter specified at (a) enables cell function to be disrupted and whereby the presence or absence of the exogenous inducer or the expression of the developmental gene promoter specified at (c) prevents or reduces the disruption of cell function.

Examples of promoter sequence responsive to the presence or absence of an exogenous inducer or a developmental gene promoters useful at (c) above include those defined above in relation to (b).

According to a fourth aspect of the present invention there is provided plant germplasm comprising a plant comprising a DNA construct as described above.

According to a fifth aspect of the present invention there is provided a mammalian cell comprising a DNA construct as described above.

According to a sixth aspect of the present invention there is provided the use of a DNA construct as described above to disrupt cell function and to optionally reverse said cell disruption.

Preferably, the first DNA sequence of the DNA construct capable of reversibly disrupting cell function further comprises an operator sequence, responsive to a repressor protein coded for by a DNA sequence comprised within a fifth DNA sequence which is operably linked and under the control of a sixth DNA sequence comprising an inducible promoter sequence, responsive to the presence or absence of an exogenous inducer. Examples of promoters suitable for use as the sixth DNA sequence include those listed above for use as the second DNA sequence.

Preferably, the fourth DNA sequence of the DNA construct which is capable of reversibly disrupting cell function comprises a DNA sequence coding for a member of a family of proteins which are naturally associated with the DNA sequence specified in (b). The fourth DNA sequence preferably comprises a DNA sequence coding for a protease propeptide. Alternatively, the protein is an enzyme inhibitor or even an antibody, raised against the product of the said second DNA sequence.

Preferably, the protease is a targeted or non-targeted cysteine protease enzyme. Examples of naturally occuring cysteine protease enzymes and nucleic acid sequences which encode them are described for instance in WO 97/35983.

Preferably, the developmental gene promoter is an early seedling/seed promoter. Alternatively, it may be a pathgen-induced promoter.

Preferably, the DNA construct is capable of disrupting cell function in a plant cell or a mammalian cell.

According to a seventh aspect of the present invention there is provided a construct comprismg:

(a) a first DNA sequence comprising either an inducible promoter sequence responsive to the presence or absence of an exogenous inducer or a developmental gene promoter capable of initiating gene expression in a selected tissue or at a selected stage of development of a plant; and

(b) a second DNA sequence comprising a DNA sequence, the product of which is capable of inhibiting a protein capable of controlling senescence in a plant operably linked and under the control of the promoter sequence specified at (a),

whereby the presence or absence of the exogenous inducer or the activity of the developmental gene promoter enables the synthesis of the protein capable of controlling senescence to be down-regulated or its activity to be substantially reduced, thereby enabling the process of senescence to be slowed down.

According to an eighth aspect of the present invention there is provided the use of a DNA construct as defined above to down-regulate the synthesis or substantially reduce the activity of a protein capable of controlling senescence.

According to a nineth aspect of the invention, there is provided plant geriplasm comprising a plant comprising a DNA construct according to the seventh aspect defined above.

Preferably, the second DNA sequence of the DNA construct which is capable of slowing down the process of senescence comprises a DNA sequence coding for a member of a family of proteins which are naturally-associated with the protein capable of controlling senescence. Preferably, the second DNA sequence comprises a DNA sequence coding for a protease propeptide. Alternatively, the DNA sequence codes for a protease partial sense or antisense RNA. In yet a further alternative, the DNA sequence codes for an antibody raised against the product of the second DNA sequence and capable of inhibiting the activity thereof.

Preferably, the protein capable of controlling senescence is a protease enzyme. The protease may be derived from plants, fungi, bacteria or animals or may be a fragment thereof or a variant of either of these which has protease activity. Most preferably, the protease is derived from plants, fungi, bacteria or animals.

Preferably, the protease is a targeted or non-targeted cysteine protease as described above.

Preferably, the developmental gene promoter is a senescence-induced promoter.

Preferably, the DNA construct is capable of down-regulating or substantially reducing the activity of the protein capable of controlling senescence in a plant cell.

According to a tenth aspect of the present invention there is provided a DNA construct comprising:

(a) a first DNA sequence comprising either an inducible promoter sequence responsive to the presence or absence of an exogenous inducer or a developmental gene promoter capable of initiating gene expression in a selected tissue or at a selected stage of development of a plant; and

(b) second DNA sequence comprising a DNA sequence, the product of which is capable of down-regulating or inhibiting a protein capable of controlling metabolism of stored protein in a plant operably linked and under the control of the promoter sequence specified at (a),

whereby the presence or absence of the exogenous inducer or the expression of the developmental gene promoter specified at (a) enables the synthesis of the protein capable of controlling metabolism of stored protein to be down-regulated or its activity to be substantially reduced, thereby altering the nature, the mobilisation and/or the distribution of stored products in plants.

In particular, such a construct can be used to prevent pre-harvest sprouting by causing germination of the plant to be delayed.

According to an eleventh aspect of the present invention there is provided a plant, plant seed or plant cell comprising a DNA construct as defined above in the tenth aspect.

According to a twelth aspect of the present invention there is provided the use of a DNA construct as defined above to down-regulate the synthesis or substantially reduce the activity of a protein capable of controlling metabolism of stored protein, for example as occurs during germination.

Preferably, the first DNA sequence of the DNA construct capable of controlling metabolism of stored protein further comprises an operator sequence responsive to a repressor protein coded for by a DNA sequence comprised within a third DNA sequence which is operably linked and under the control of fourth DNA sequence comprising an inducible promoter sequence responsive to the presence or absence of an exogenous inducer, whereby expression of the repressor protein prevents modification of the metabolism of stored protein.

In this way, the effects of the construct on the metabolism of stored protein can be reversed.

Alternatively or preferably additionally, the DNA construct also comprises a fifth DNA sequence comprising a DNA sequence coding for a heterologous protein which is operably linked and under the control of a sixth DNA sequence comprising an inducible promoter sequence, responsive to the presence or absence of an exogenous inducer whereby expression of the heterologous gene halts modification to stored protein metabolism.

Preferably, the protein capable of controlling metabolism of stored protein, which is capable of being inhibited by the product encoded by the DNA sequence comprised within the DNA construct in a plant, is a protease enzyme, suitably an endogenous protease enzyme. Preferably, it is a targeted or non-targeted cysteine protease enzyme as described above.

Preferably, the second DNA sequence of the DNA construct which is capable of modifying metabolism of stored protein in a plant comprises a DNA sequence coding for a product which is naturally associated with the protein capable of modifying metabolism of stored protein.

Preferably, the second DNA sequence comprises a DNA sequence coding for a protease propeptide.

Preferably, the second DNA sequence comprises a DNA sequence coding for a protease sense RNA or partial sense RNA or a DNA sequence coding for a protease antisense RNA. Alternatively, it may code for an antibody which is specific for and inhibits the protein capable of modifying metabolism of stored protein.

Proteases may be derived from plants, fingi, bacteria or animals or may comprise fragments of these or variants of either of these which have protease activity. Most preferably the protease is derived from a plant, fingi, bacteria or animal.

Preferably, the second DNA sequence comprises a DNA sequence coding for a cysteine protease sense, partial sense or antisense RNA.

Preferably, the heterologous protein is a protease.

Preferably, in this case, the developmental gene promoter is an early seedling promoter.

Preferably, the DNA construct is capable of down-regulating the synthesis or substantially reducing the activity of the protein capable of modifying metabolism of stored protein in plants.

According to a preferred embodiment of the present invention there is provided a DNA construct capable of reversibly disrupting cell function as defined above wherein the protein capable of disrupting cell function is a cysteine protease enzyme and wherein the protein capable of inhibiting said cysteine protease enzyme is a protease propeptide.

According to a further preferred embodiment of the present invention there is provided a DNA construct capable of slowing down or inhibiting senescence as defined above wherein the protein capable of controlling senescence is a mature cysteine protease enzyme and wherein the protein capable of inhibiting said cysteine protease enzyme is a cysteine protease propeptide.

According to another preferred embodiment of the present invention there is provided a DNA construct capable of modifying metabolism of stored protein as defined above wherein the protein capable of modifying metabolism of stored protein is a cysteine protease enzyme and wherein the said cysteine protease enzyme is inhibited by a DNA sequence coding for either a full or partial antisense or partial sense RNA of said cysteine protease enzyme.

The term “DNA construct”—which is synonymous with term such as “cassette”, “hybrid” and “conjugate”—includes DNA sequences directly or indirectly attached to one another, such as to form a cassette. An example of an indirect attachment is the provision of a suitable spacer group such as an intron sequence, intermediate each DNA sequence. The DNA sequences may furthermore be on different vectors and are therefore not necessarily located on the same vector.

The term “naturally-associated” includes complete or partial precursor proteins of the protein of interest or proteins which would bind in vivo to the protein of interest. These may be naturally occurring or synthetic.

The term “precursor protein” includes proteins which are formed prior to and converted into the protein of specific interest and includes pre-pro-protein and pro-protein organisations i.e. proteins comprising a mature enzyme region, a propeptide region and/or a target region.

The term “protein” includes polypeptides comprising one or more chains of amino acids joined covalently trough peptide bonds, oligopeptides comprising three or more amino acids covalently linked through peptide bonds and peptides consisting of two or more amino acids linked covalently through peptide bonds.

The term “protease” includes a pre-pro-enzyme, a pro-enzyme or a mature enzyme which are able to hydrolyse peptide bonds in proteins and peptides. Such proteases may be derived from plants, fungi, bacteria or animals. An example of a protease useful in the present invention is a cysteine protease (CP).

The term “product” includes a protein, a precursor protein and antisense or partial sense RNA to a DNA sequence capable of performing the stated function.

The DNA sequences of the present invention may be genomic DNA sequences which are in an isolated form and are, preferably, operably linked to DNA sequences with which they are not naturally associated, or the DNA may be synthetic DNA or cDNA.

The present invention also provides a genetically transformed organism such as a plant and parts thereof, such as cell protoplasts and seeds, having incorporated, preferably stably incorporated, into the genome of the organism the DNA constructs of the present invention. Thus, the present invention provides an organism the cells of which can be reversibly inhibited at an appropriate developmental stage in which the organism contains, preferably stably incorporated in its genome, the recombinant DNA construct as defined above. In this regard, the protein capable of disrupting cell function can be detargeted (i.e. to become cytosolic) or retargeted to a specific area of the cell such as to mitochondria or chloroplasts. The disruption of cell function can then be reversed by the expression of a protein which specifically inhibits the protein disrupting cell function.

An advantage of the present invention is that the protein capable of disrupting cell function is specifically inhibited by a protein encoded by a DNA sequence comprised within the DNA construct. In addition, for two of the applications, the inhibition takes place at the protein level rather than at the DNA level. Therefore any cytotoxic protein synthesized and accumulated prior the onset of repression will also be inhibited.

The present invention also provides a plant, in which the synthesis of a protein capable of controlling senescence is down-regulated by expression of a protein which specifically inhibits it.

An advantage of the DNA constructs capable of slowing down or inhibiting senescence of the present invention is that in so doing the yield of the plant in which it incorporated is increased. Further details on the regulation of senescence are given in our International Patent Application No. WO 95/07993 which is incorporated herein by reference.

The suppression of the proteins involved in senescence can be controlled by the use of either “antisense” or “partial-sense” technology.

A DNA construct according to the present invention may be an “antisense” construct generating “antisense” RNA or a “partial-sense” construct (encoding at least part of the functional gene product) generating “partial-sense” RNA.

“Antisense RNA” is an RNA sequence which is complementary to a sequence of bases in the corresponding mRNA: complementary in the sense that each base (or the majority of bases) in the antisense sequence (read in the 3′ to 5′ sense) is capable of pairing with the corresponding base (G with C, A with U) in the mRNA sequence read in the 5′ to 3′ sense. Such antisense RNA may be produced in the cell by transformation with an appropriate DNA construct arranged to generate a transcript with at least part of its sequence complementary to at least part of the coding strand of the relevant gene (or of a DNA sequence showing substantial homology therewith).

“Partial-sense RNA” is an RNA sequence which is substantially homologous to at least part of the corresponding mRNA sequence. Such partial-sense RNA may be produced in the cell by transformation with an appropriate DNA construct arranged in the normal orientation so as to generate a transcript with a sequence identical to at least part of the coding strand of the relevant gene (or of a DNA sequence showing substantial homology therewith) without the ability to encode a functional protein. Suitable partial-sense constructs may be used to inhibit gene expression (as described in International Patent Publication WO91/08299).

The suppression of the proteins involved in senescence can alternatively be controlled by the use of the propeptide of a protease involved in the senescence process. Over-expressing a propeptide alone would inhibit a mature protease thus delaying senescence

Other approaches which are, or become, available may also be used.

The DNA constructs of the present invention may advantageously be used to optimise plant performance and yield.

As a route to modifying storage protein content, nature or localisation in plants we propose the selective down-regulation of proteins such as cysteine proteases throughout the plant (Mino and Inoue, 1988). This may be achieved through the use of “sense” or “antisense” technology. A beneficial effect might, therefore, be obtained through partial sense or anti-sense down-regulation of these proteases in seed, cotyledons, roots or stems. A number of other key enzymes have also been described to play a role in storage protein mobilisation and/or translocation. The down-regulation of these genes using the same partial sense or anti-sense strategies might also be useful in the present invention to improve plant performance.

The inhibition of these proteins may also be achieved by the use of a precursor protein of the protein to be inhibited.

To prevent or reverse storage protein metabolism to be altered, the synthesis of the partial-sense or anti-sense could be repressed using a repressor/operator strategy in combination with an inducible promoter. Alternatively, an heterologous protease gene the to sequence of which differs enough from the endogenous protease gene could be expressed under the control of an inducible promoter. This would avoid the occurrence of any down-regulation by the partial-sense or anti-sense.

During seed maturation and storage the seed promoter would drive, for example, partial sense to a cysteine protease, and so inhibit protein degradation and thus reduce the loss of storage proteins and the incidence of pre-harvest sprouting.

It will be appreciated that the use of the developmental gene promoter of the present invention restricts expression of the sequence to which it is operatively linked to a suitable stage of plant development, and also means that it is not necessary to continue to apply an exogenous inducer to the plant throughout its lifetime. This has both economic and ecological benefits.

Cysteine proteases are synthesised as an inactive precursor comprising a propeptide fused to the mature enzyme. The propeptide seems to inhibit the mature enzyme by folding on it. Furthermore, the propeptide alone, expressed in E. coli and purified, shows inhibition of the mature enzyme in vitro (Taylor et al., 1995). Inhibition of cysteine proteases could therefore be carried out by expressing the pro-peptide under the control of an inducible or stage specific, resulting in a reduction of protein loss. The physiological effect may be reversed by down-regulating the synthesis of the pro-peptide using a repressor/operator strategy or by expressing an heterologous protease which sequence differs enough from the endogenous one to avoid any binding of the propeptide.

As a further strategy to obtain cell death/inhibition, system, re-targeted cysteine protease could be expressed, for example targeted to the mitochondria or chloroplasts. Expression of a cysteine protease in a different cellular compartment may have inhibitory results. This protease could be fused to a chloroplast or a mitochondrial targeting sequence, such as the pre-B from Nicotiana plumbaginifolia (Boutry et al., [1987], Nature, 328, 341), therefore achieving protein degradation in these sensitive organelles. CPs could also be retargeted to the cytoplasm or to the endoplasmic reticulum, fused or not to an ER retention signal.

Since CPs have been shown to be associated with cell autolysis in plants (Minami and Fukuda, 1995), an alternative approach would be to express cysteine proteases with no targeting sequences, so achieving cytosolic expression. Again this may cause protein degradation and cell death.

The DNA constructs of the present invention are introduced into a plant by transformation. The method employed for transformation of the plant cells is not especially germane to this invention and any method suitable for the target plant may be employed. Transgenic plants are obtained by regeneration from the transformed cells. Numerous transformation procedures are known from the literature such as agroinfection using Agrobacterium tumefaciens or its Ti plasmid, electroporation, microinjection or plants cells and protoplasts, microprojectile transformation, to mention but a few. Reference may be made to the literature for fill details of the known methods.

Neither is the plant species into which the DNA construct is inserted particularly germane to the invention. Dicotyledonous and monocotyledonous plants can be transformed. This invention may be applied to any plant for which transformation techniques are, or become, available. The DNA constructs of the present invention can therefore be used in a variety of genetically modified plants, including field crops such as canola, sunflower, tobacco, sugarbeet, and cotton; cereals such as wheat, barley, rice, maize, and sorghum; fruit such as tomatoes, mangoes, peaches, apples, pears, strawberries, bananas and melons; and vegetables such as carrot, lettuce, cabbage and onion. The promoter is also suitable for use in a variety of tissues, including roots, leaves, stems and reproductive tissues.

We have also isolated and characterised nucleic acid sequences which code for novel cysteine proteases and these are described in our co-pending International Patent Application No. WO97/35983.

The present invention will now be described only by way of non-limiting example with reference to the accompanying drawings, in which:

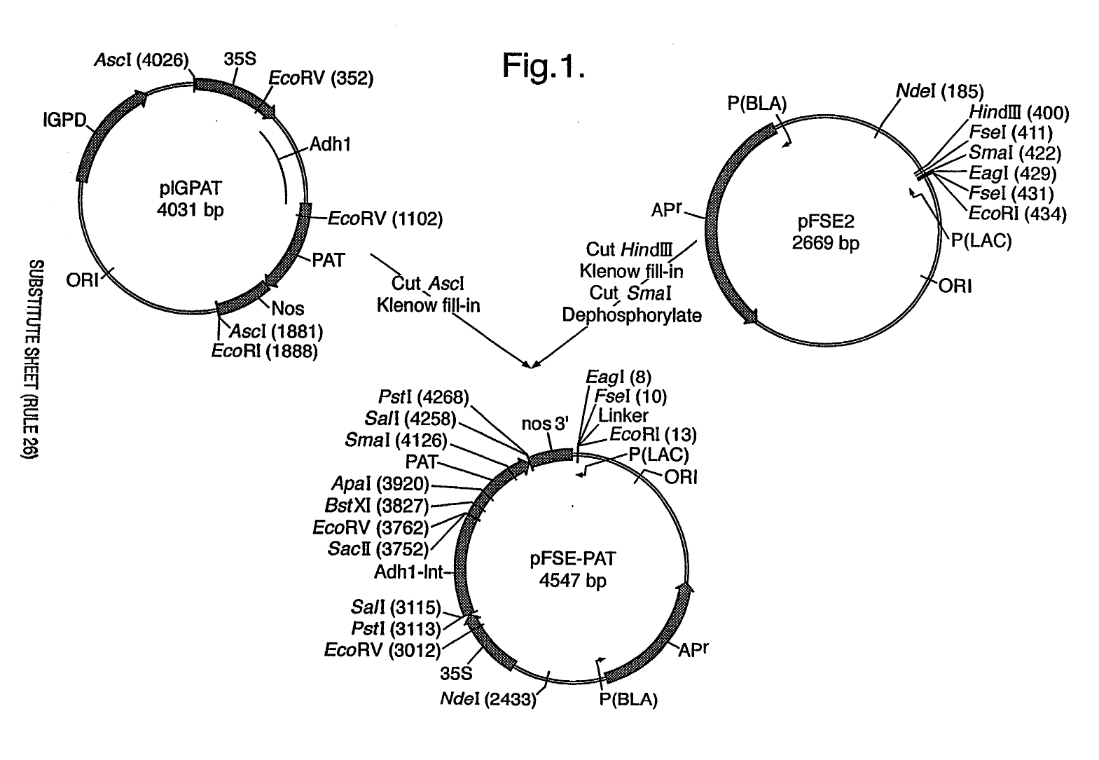

FIG. 1 shows a schematic outline of the construction of maize transformation vector, pFSE-PAT

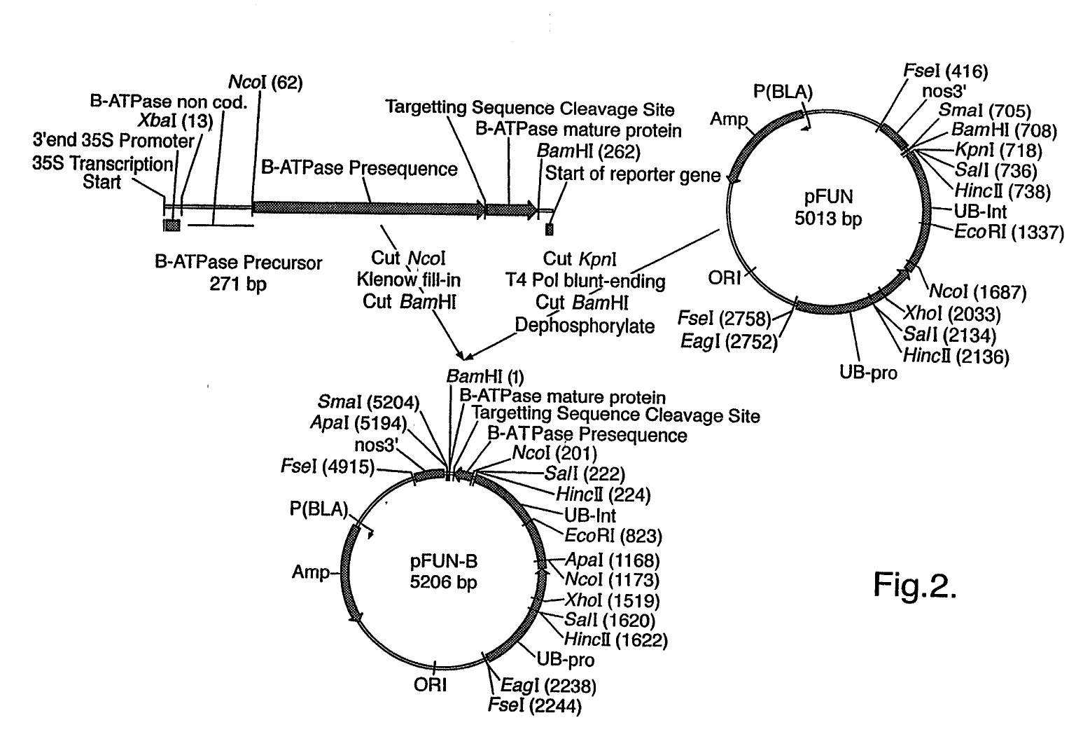

FIG. 2 shows a schematic outline of the construction of vector, pFUN-β.

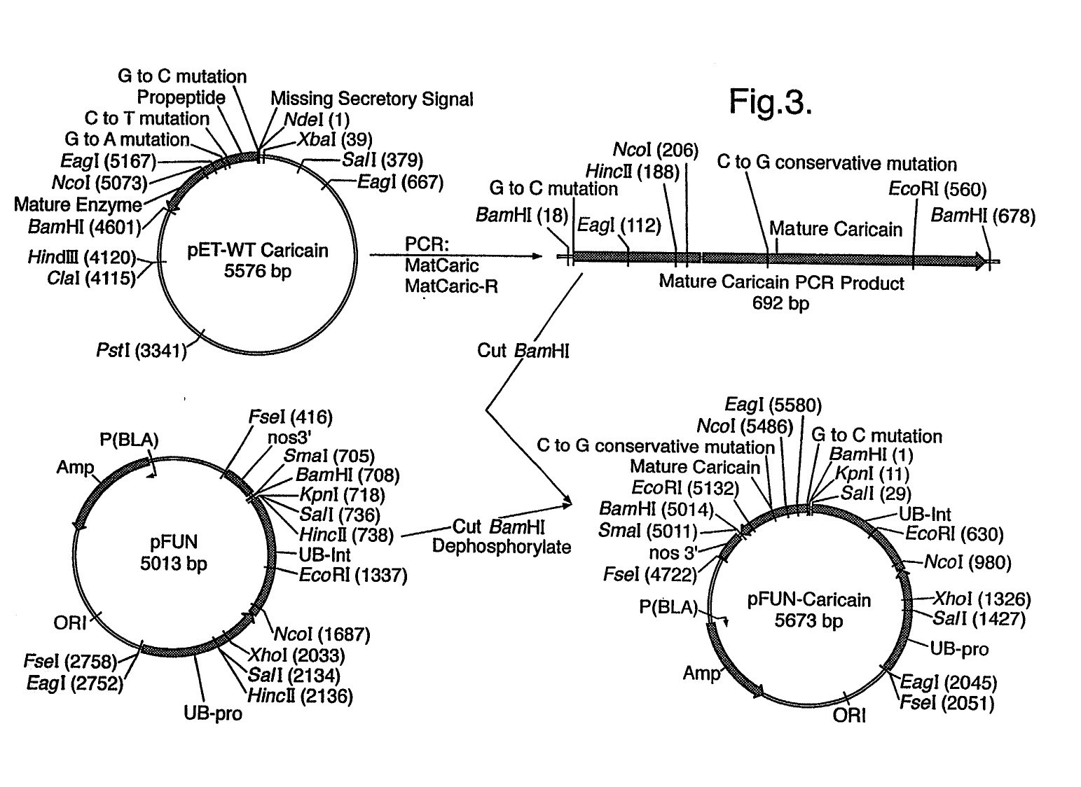

FIG. 3 shows a schematic outline of the construction of maize transformation vector, pFUN-Caricain.

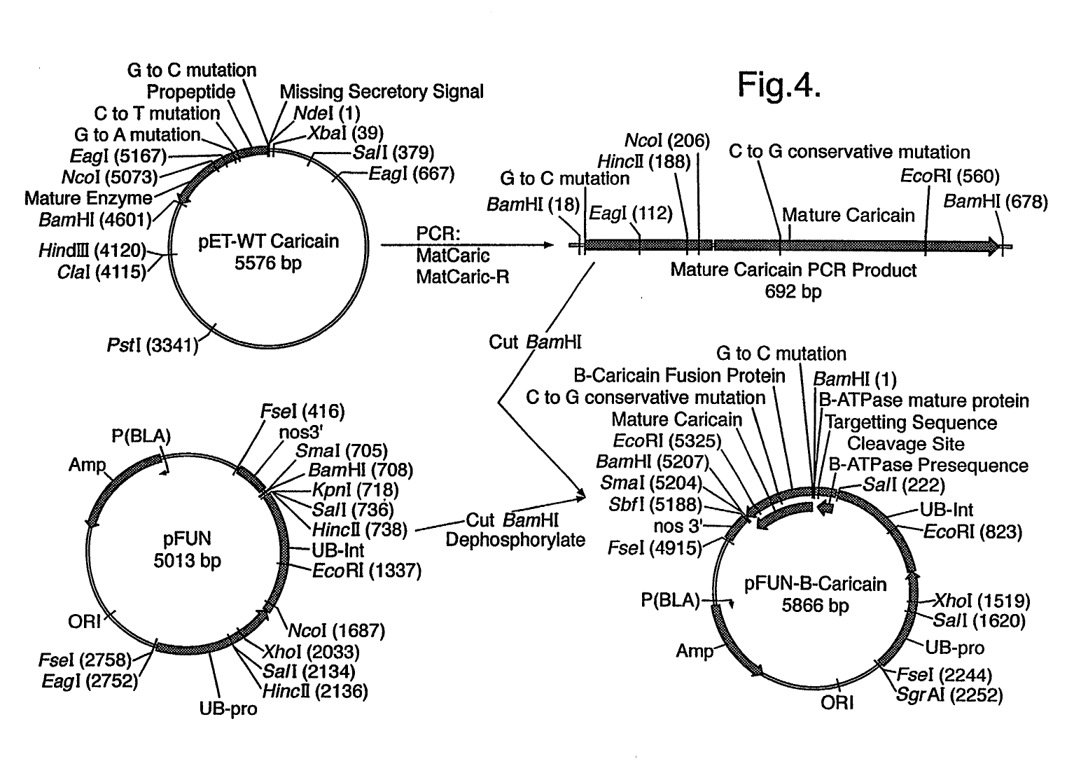

FIG. 4 shows a schematic outline of the construction of maize transformation vector, pFUN-β-Caricain.

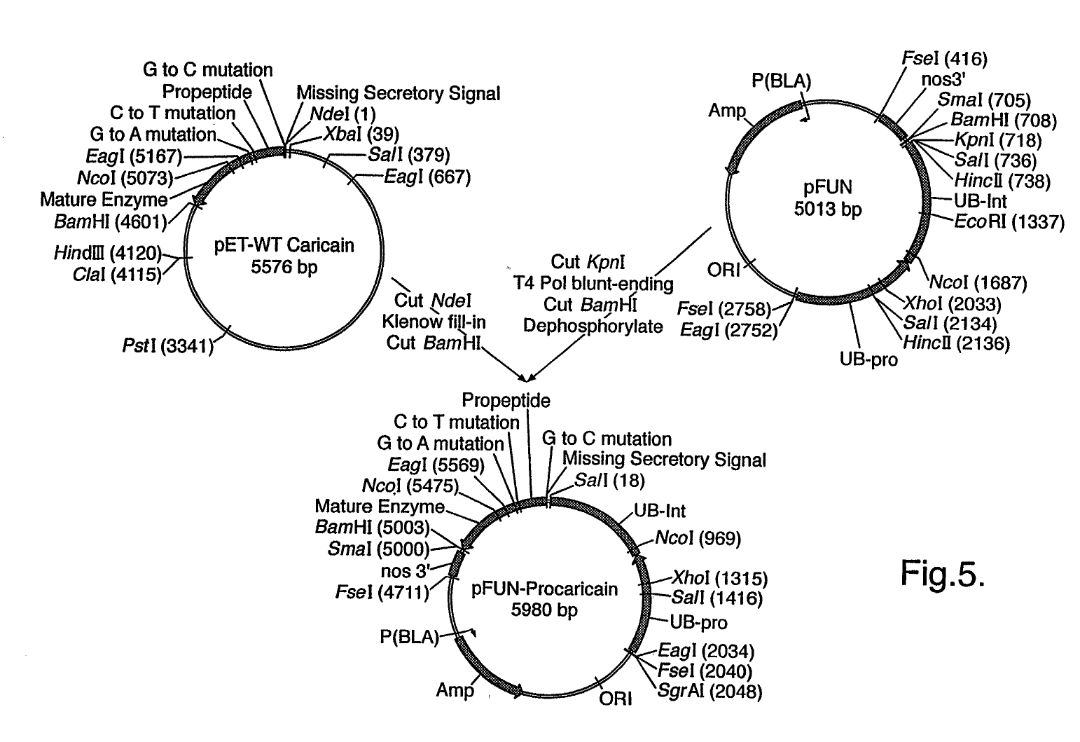

FIG. 5 shows a schematic outline of the construction of maize transformation vector, pFUN-Procaricain.

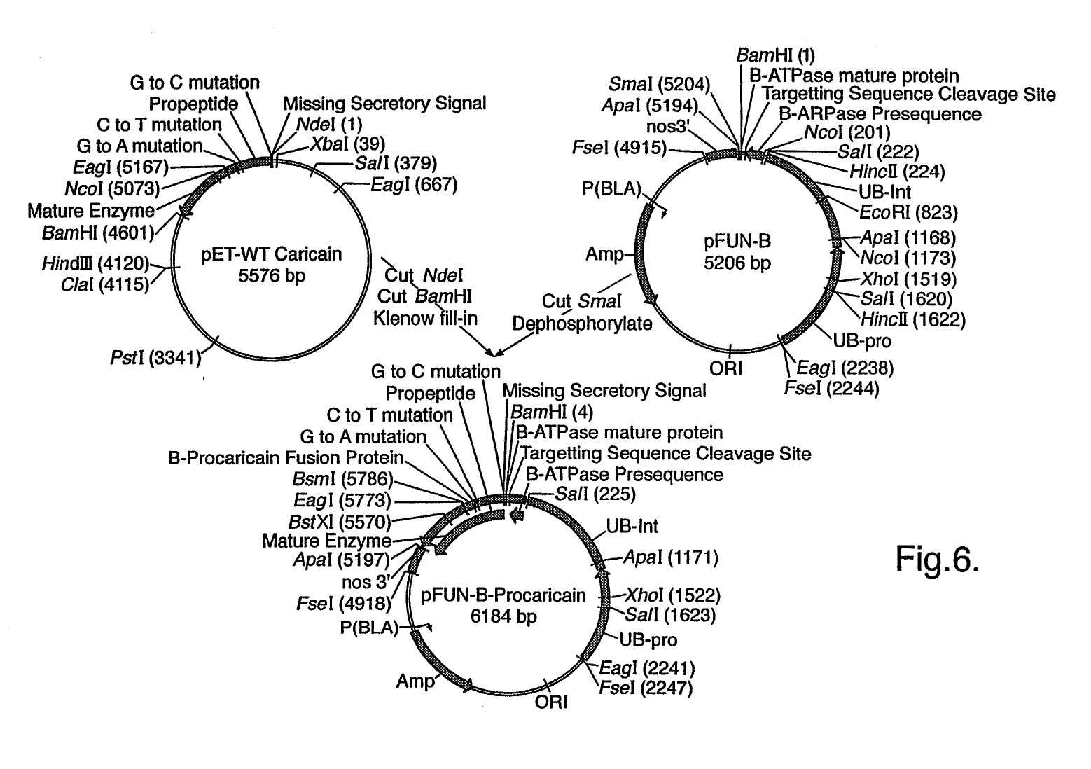

FIG. 6 shows a schematic outline of the construction of maize transformation vector, pFUN-β-Procaricain.

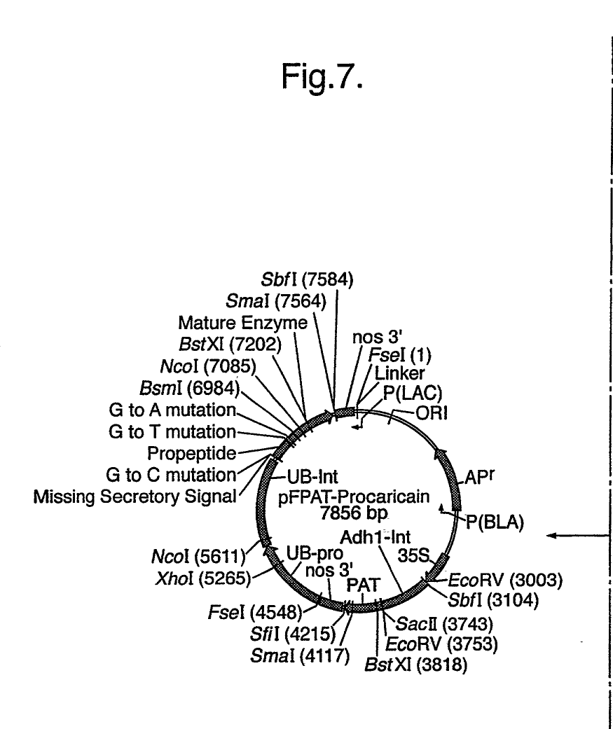

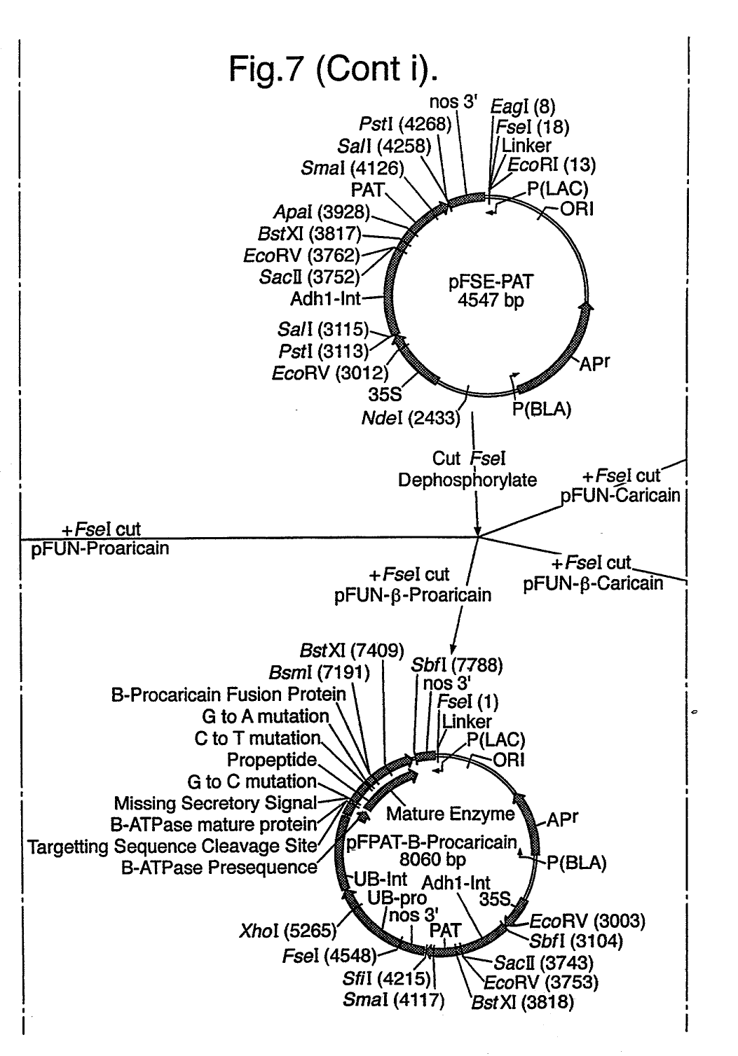

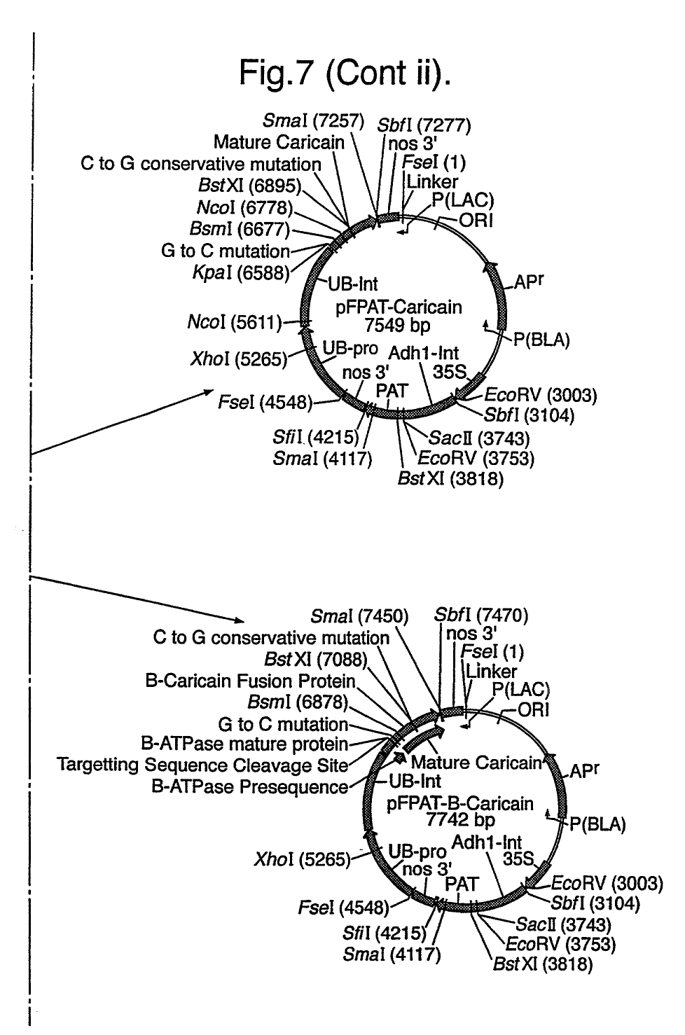

FIG. 7 shows a schematic outline of the construction of maize transformation vectors, pFPAT-Caricain, pFPAT-β-Caricain, pFPAT-Procaricain and pFPAT-β-Procaricain

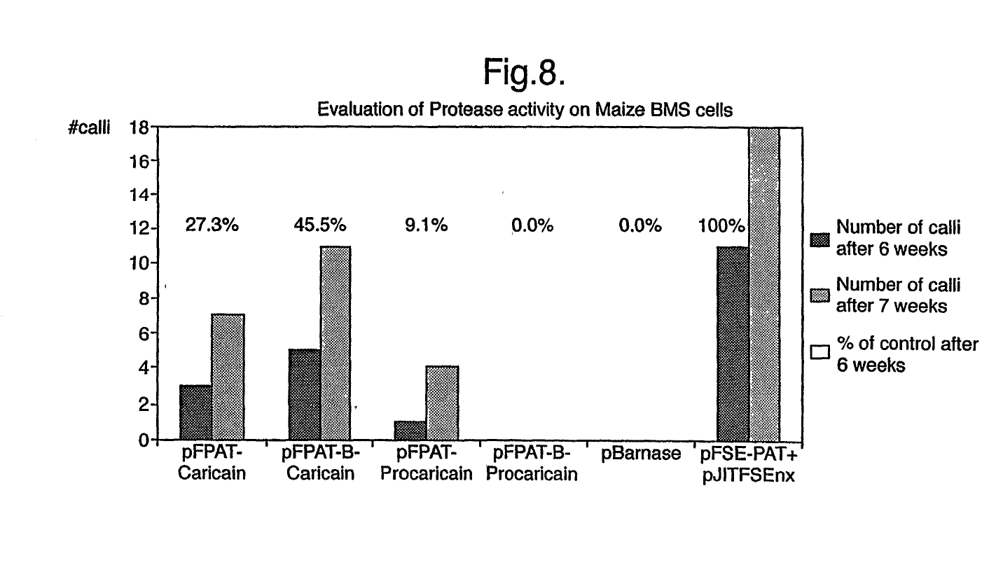

FIG. 8 illustrates that cysteine proteases can inhibit plant cell regeneration.

### EXAMPLE 1

Over-expression of CPs in Corn Calli to Demonstrate Their Potential as Inhibitory Genes

Aim

The objective of this experiment was to show that expression of a de-targeted or re-targeted protease in cultured BMS corn cells results in a reduction of cell viability as measured by the establishment of transgenic calli following transformation, in comparison with the establishment of calli transformed with a vector not containing the inhibitory cassette, thereby indicating that the expression of a protease in the cytosol or in the mitochondria is inhibiting cell growth. As an example we chose caricain (or proteinase omega), a cysteine protease from Carica papaya (EMBL: X66060). The targeting sequence of the pre-pro-enzyme was removed in order to prevent its entry to the secretory pathway. As a result the protease should be expressed in the cytoplasm as a proenzyme (pFPAT-Procaricain) or a mature enzyme (pFPAT-Caricain). In addition, the protease was re-targeted to the mitochondria, again as a proenzyme pFPAT-β-Procaricain) or a mature enzyme (pFPAT-β-Caricain).

Construction of the Maize Transformation Vectors

pFPAT-Caricain, pFPAT-β-Caricain, pFPAT-Procaricain and pFPAT-β-Procaricain are plant transformation vectors in which the expression of caricain partial or full length cDNA is under the control of the maize ubiquitin promoter (Ub-pro) fused to its intron (UB-int) and the terminator of the Agrobacterium tumefaciens nopaline synthase gene. In addition, the vectors contain a “PAT cassette”, conferring resistance to the herbicide Basta to the transformed cells. A schematic outline of the construction of the maize transformation vectors pFPAT-Caricain, pFPAT-β-Caricain, pFPAT-Procaricain and pFPAT-β-Procaricain is given in FIGS. 1-6.

The “PAT cassette” was excised as an AscI fragment from pIG-PAT and filled-in using Klenow polymerase. The generic maize transformation vector pFSE-PAT was then created by inserting the “PAT cassette” into a pFSE2 vector digested by SmaI and HindIII (to remove one of the two FseI site) and filled-in using Klenow polymerase (FIG. 1).

Effector cassettes can be inserted using the unique FseI site.

A targeting sequence for plant mitochondria was obtained from a cDNA clone encoding the β-subunit of an ATPase gene isolated from Nicotiana plumbaginifolia (Chaumont et al., 1994). The targeting sequence fragment was obtained by a first digest of the cDNA clone with NcoI, a filling-in reaction using Klenow polymerase and a subsequent digest with BamHI. The sequence was then ligated into the pFUN vector digested with KpnI, blunt-ended using T4 DNA Polymerase and subsequently digested with BamHI, to create PFUN-β (FIG. 2).

The mature caricain-encoding fragment was obtained by PCR on pET-WT-Caricain using a forward oligonucleotide, MatCaric (5′GTTTATTAATGAAGATGGATCCATGCTGCCCGAGAAT3′), modified to introduce a BamHI site and an optimised translation start at the 5′end of the mature sequence, and a reverse oligonucleotide, MatCaric-R (5′GTTAGCAGCCGGATCCTCAATTTTTA3′). The PCR fragment was then digested with BamHI and ligated into the pFUN and PFUN-β vectors also digested with BamHI, to create PFUN-Caricain (FIG. 3) and PFUN-β-Caricain (FIG. 4) respectively.

A procaricain-encoding fragment was obtained by a digest of pET-WT-Caricain with NdeI, a filling-in reaction using Klenow polymerase and a further digest with BamHI. The DNA fragment was then ligated into the pFUN vector digested with KpnI, blunt-ended using T4 DNA Polymerase and further digested with BamHI, to create PFUN-Procaricain (FIG. 5).

In addition, the procaricain-encoding fragment was obtained by simultaneous digest of pET-WT-Caricain with NdeI and BamHI, followed by a filling in reaction using Klenow polymerase. The DNA fragment was then ligated into the PFUN-β vector digested with SmaI to create PFUN-β-Procaricain (FIG. 6).

FseI cassettes containing procaricain or mature caricain DNA driven by the maize polyubiquitin promoter (UBI) and its intron and followed by the nos3′ terminator were excised from pFUN-Caricain, pFUN-β-Caricain, pFUN-Procaricain and pFUN-β-Procaricain. The FseI cassettes were then cloned into the FseI site of pFSE-PAT to generate the plant transformation vectors pFPAT-Caricain, pFPAT-β-Caricain, pFPAT-Procaricain and pFPAT-β-Procaricain respectively (FIG. 7).

Transformation of BMS Corn Cells

pFPAT-Caricain, pFPAT-β-Caricain, pFPAT-Procaricain and pFPAT-β-Procaricain were transformed into maize BMS cells. A double control was obtained by co-transforming BMS cells with pFSE-PAT, which contains the PAT selection cassette alone, and pJITFSEnx. Vector pJITFSEnx consists of a double enhanced CAMV 35S promoter driving the GUS reporter gene and does not include any PAT selection cassette. This combination of vectors was used as a negative control as it should not inhibit calli formation. Since cells transformed with pJITFSEnx alone will not regenerate on bialophos, it was also used as a co-transformation control. The frequency of co-transformation that can be achieved using BMS wisker transformation is important for subsequent experiments (example 2) and it was estimated by counting the proportion of regenerating calli that also express GUS (double transformants).

A positive control was provided by pBarnase which contains a PAT selection cassette followed by the potent cytotoxic ribonuclease gene barnase driven by the CAMV 35S promoter. This construct was anticipated to strongly reduce the successful establishment of transgenic calli.

All transformation experiments were performed in triplicate and the results pooled together.

The transformation vectors were introduced into cultured BMS cells using,a silicon carbide fibre-mediated transformation technique as follows:

Preparation of Silicon Carbide Whiskers

Dry whiskers were always handled in a fume cabinet, to prevent inhalation and possible lung damage. These whiskers may be carcinogenic as they have similar properties to asbestos. The Silar SC-9 whiskers were provided by the Advanced Composite Material Corporation Greer, S.C., USA. The sterile whisker suspensions were prepared in advance as follows. Approximately 50 mg of whiskers were deposited into a pre-weighed 1.5 ml Eppendorf tube, which was capped and reweighed to determine the weight of the whiskers. The cap of the tube was perforated with a syringe needle and covered with a double layer of aluminium foil. The tube was autoclaved (121° C., 15 psi, for 20 minutes) and dried. Fresh whisker suspensions were prepared for each experiment, as it had been reported that the level of DNA transformation when using fresh suspensions was higher than that of older suspensions. A 5% (weight/volume) whisker suspension was prepared using sterile deionised water. This was vortexed for a few seconds to suspend the whiskers immediately before use.

DNA Transformation into Cells

All procedures were carried out in a laminar air flow cabinet under aseptic conditions. The DNA was transformed into the cells using the following approach. Specific modifications to this method are indicated in the text. Cell and whisker suspensions were pipetted using cut down Gilson pipette tips. 100 μl of fresh BMS medium was measured into a sterile Eppendorf tube. To this was added 40 μl of the 5% (w/v) whisker suspension and 10 μl (1 mg/ml) of the plasmid DNA, which was vortexed at top speed for 60 seconds using a desktop vortex unit (Vortex Genie 2 Scientific Industries, Inc). Immediately after this period of vortexing, 500 μl of the cell suspension was added ie 250 μl of packed cells. The Eppendorf tube was then capped and vortexed at top speed for 60 seconds in an upright position. The same procedure was used to transform the other cell lines.

Results

As shown in FIG. 8, all protease constructs resulted in a decrease in the number of calli regenerating on media containing bialophos, indicating that CPs might be detrimental to cell growth. pFPAT-B-Procaricain, that targets the CP precursor to mitochdria is as potent as barnase in the conditions of the experiment and totally prevents maize calli establishment. Similarly, the integration of pFPAT-Procaricain, pFPAT-Caricain and pFPAT-β-Caricain into the BMS cells genome leads to a reduction in the number of calli regenerating, with figures totalising 9.1%, 27.3% and 45.5% of the control respectively, 6 weeks after transformation.

The higher activity of the precursor-enzyme constructs compared to the mature enzymes is interesting since precursor proteins must loose their propeptide in order to be activated. However, caricain was specifically chosen for exemplification because its propeptide is very labile and a substantial proportion of it is thought to be cleaved in a self-catalytic reaction, even at neutral pH (Goodenough P W, personal communication). The lower activity of the mature enzyme constructs could be attributed to a folding problem since the amino-terminal propeptide is thought to act as a scaffolding that would allow the mature enzyme to take its correct conformation (Taylor et al., 1995).

Although those results are preliminary, they have already been confirmed by a similar experiment undertaken on a wheat cell suspension system that can not be disclosed to date. All experiments are in the process to be repeated.

### EXAMPLE 2

Over-expression of Cysteine Protease Propeptides in Corn Calli to Demonstrate Their Potential to Inhibit the Corresponding Mature Enzyme

Aim

The objective of this experiment was to show that simultaneous expression of the protease and its cognate propeptide in cultured BMS corn cells results in an increased cell viability compared to the expression of the protease alone, as measured by the establishment of transgenic calli following transformation, thereby indicating that the expression of the propeptide is inhibiting the mature enzyme in plants.

Construction of the Maize Transformation Vectors pFUN-Propeptide and pFUN-βPropeptide

The 5′-end region of the caricain cDNA, encoding the propeptide area, was amplified by PCR on pET-WT-Procaricain using modified primers that introduce a BamHI site at both ends of the new molecule. The PCR fragment was digested by BamHI and subcloned into both the pFUN and the pFUN-β vectors also digested by BamHI (between the polyubiquitin intron and the nos 3′ terminator), to generate pFUN-Propeptide and pFUN-β-Propeptide respectively.

Transformation of BMS Corn Cells

The pFPAT-Caricain and pFPAT-Procaricain DNA vectors were respectively co-transformed into cultured BMS cells together with increasing amounts of the pFUN-Propeptide DNA vector. Similarly, the pFPAT-β-Caricain and pFPAT-β-Procaricain DNA vectors were respectively co-transformed into cultured BMS cells together with increasing amounts of the pFUN-β-Propeptide DNA vector. All transformations were performed using the silicon carbide fibre-mediated transformation technique described in Example 1. The positive, negative and co-transformation controls described in Example 1 were also included in the experiment.

Other modifications of the present invention will be apparent to those skilled in the art without departing from the scope of the invention.

References

Ascenzi P, Aducci P, Torroni A, Amiconi G, Ballio A, Menegatti E, Guarneri M: The pH dependence of pre-steady-state and steady-state kinetics for the papain-catalyzed hydrolysis of n-alpha carbobenzoxyglycine p-nitrophenyl ester. Biochem Biophys Acta 912 (2): 203-210 (1987).

Becker C, Fischer J, Nong V H, Munitz K: PCR cloning and expression analysis of cDNAs encoding cysteine proteases from germinating seeds of Vicia sativa L. Plant Mol Biol 26: 1207-1212 (1994).

Bevan, M: Binary Agrobacterium vectors for plant transformation. Nucleic Acids Res 12, 8711-8721 (1984).

Boylan M T, Sussex I M: Purification of an endopeptidase involved with storage-protein degradation in Phaseolus vulgaris L. cotyledons. Planta 170 (3): 343-352 (1987).

Bradford M M: A rapid and sensitive method for the quantification of microgram quantities of protein utilising the principle of protein-dye binding. Anal Biochem 72: 248-254 (1976).

Cammue B P A, De Bolle M F C, Terras F R G, Proost P, Van Damme J, Rees S B, Vanderleyden J, Broekaert W F: Isolation and characterisation of a novel class of plant antimicrobial peptides from Mirabilis jalapa L. Seeds. JBC 267 (4): 2228-2233 (1992).

Cervantes E, Rodriguez A, Nicolas G: Ethylene regulates the expression of a cysteine protease gene during germination of chickpea (Cicer arietinum L.). Plant Mol Biol 25 (2): 207-215 (1994).

Cercos M, Mikkonen A, Ho T-H D: Promoter analysis of a GA-induced cysteine endoprotease gene in barley aleurone cells. Plant Physiol (Rockville) 108 (2 SUPPL.): 79 (1995).

Cohen L W, Coghlan V M, Dihel L C: Cloning and sequencing of papain-encoding cDNA. Gene 48: 219-227 (1986).

Comai L, Harada J J: Transcriptionnal activities in dry seed nuclei indicate the timing of the transition from embryogeny to germination. Proc Natl Acad Sci USA 87: 2671-2674 (1990).

deBarros E G, Larkins B A: Cloning of a cDNA encoding a putative cysteine protease from germinating maize seeds. Plant Science 99 (2): 189-197 (1994).

Dietrich R A, Maslyar D J, Heupel R C, Harada J J: Spatial patterns of gene expression in Brassica-napus seedlings: identification of a cortex-specific gene and localization of messenger RNA encoding isocitrate lyase and a polypeptide homologous to proteases. Plant Cell 1(1): 73-80 (1989).

Edwards K, Johnstone C, Thompson C: A simple and rapid method for the preparation of plant genomic DNA for PCR analysis. Nucleic Acids Res 19 (6): 1349 (1991).

Goetting-Minesky M P, Mullin B C: Differential gene expression in an actinorhizal symbiosis: Evidence for a nodule-specific cysteine protease. Proc Natl Acad Sci USA 91 (21): 9891-9895 (1994).

Goetting-Minesky P, Mullin B C: Evidence for a nodule-specific cysteine protease in an actinorhizal symbiosis. American Journal of Botany 81 (6 SUPPL.): 24 (1994).

Graham I A, Baker C J, Leaver C J: Analysis of the cucumber malate synthase gene promoter by transient expression and gel retardation assays. Plant Journal 6 (6): 893-902 (1994).

Grossberger: Minipreps of DNA from bacteriophage lambda. Nucleic Acids Res 15 (16): 6737 (1987).

Guerrero F D, Jones J T, Mullet J E: Turgor-responsive gene transcription and RNA levels increase rapidly when pea shoots are wilted sequence and expression of three inducible genes. Plant Mol Biol 15 (1): 11-26 (1990).

Hara-Nishimura I, Takeuchi Y, Nishimura M: Molecular characterization of a vacuolar processing enzyme related to a putative cysteine protease of Schistosoma mansoni. Plant Cell 5 (11): 1651-1659 (1993).

Hara-Nishimura I, Shimada T, Hiraiwa N, Nishimura M: Vacuolar processing enzyme responsible for maturation of seed proteins. Journal of Plant Physiology 145 (5-6): 632-640 (1995).

Holsters M, de Waele D, Depicker A, Messen E, Van Montagu M, Schell J: Transfection and transformation of A. tumefaciens. Mol Gen Genet 163: 181-187 (1987).

Jefferson R A, Bevan M, Kavanagh T: The use of the Escherichia coli beta-glucuronidase gene as a gene fusion marker for studies of gene expression in higher plants—construction of promoter cloning vector plasmid pTAKI and detection of enzyme activity by fluorometric assay. Biochem Soc Trans 15: 17-18 (1987).

Jepson I, Bray J, Jenkins G, Schuch W, Edwards K: A rapid procedure for the construction of PCR cDNA libraries from small amounts of plant tissue. Plant Mol Biol Reporter 9 (2): 131-138 (1991).

Jiang B, Siregar U, Willeford K O, Luthe D S, Williams W P: Association of a 33-kilodalton cysteine protease found in corn callus with the inhibition of fall armyworm larval growth. Plant Physiol 108 (4): 1631-1640 (1995).

Jones M L, Larsen P B, Woodson W R: Ethylene-regulated expression of a carnation cysteine protease during flower petal senescence. Plant Mol Biol 28 (3): 505-512 (1995).

Karrer K M, Peiffer S L, DiTomas M E: Two distinct gene subfamilies within the family of cysteine protease genes. Proc Natl Acad Sci USA 90: 3063-3067 (1993).

Kianian S F, Quiros C F: Genetic analysis of major multigene families in Brassica oleracea and related species. Genome 35 (3): 516-527 (1992).

Koehler S M, Ho T-H D: Hormonal regulation processing and secretion of cysteine proteases in barley aleurone layers. Plant Cell 2 (8): 769-784 (1990).

Korodi I; Asboth B; Polgar L: Disulfide bond formation between the active-site thiol and one of the several free thiol groups of chymopapain. Biochemistry 25 (22): 6895-6900 (1986).

Lidgett A, Moran M, Wong K A L Furze J, Rhodes M J C and Hamill J D: Isolation and expression pattern of a cDNA encoding a cathepsin B-like protease from Nicotiana rustica. Plant Mol Biol 29 (2): 379-384 (1995).

Lin E; Burns D J W; Gardner R C: Fruit developmental regulation of the kiwifruit actinidin promoter is conserved in transgenic petunia plants. Plant Mol Biol 23 (3): 489-499 (1993).

Linthorst H J M, Van Der Does C, Van Kan J A L, Bol J F: Nucleotide sequence of a cdna clone encoding tomato Lycopersicon esculentum cysteine protease. Plant Physiol 101 (2): 705-706 (1993).

Linthorst H J M, Van Der Does C, Brederode F T, Bol J F: Circadian expression and induction by wounding of tobacco genes for cysteine protease. Plant Mol Biol 21 (4): 685-694 (1993).

Mallinson D J, Lockwood B C, Coombs G H, North M J: Identification and molecular cloning of four cysteine protease genes from the pathogenic protozoon Trichomonas vaginalis. Microbiology 140 (10): 2725-2735 (1994).

Mikkelsen T R, Andersen B and Jorgensen R B: The risk of crop transgene spread. Nature 380: 31 (Mar. 7, 1996).

Marttila S, Porali I, Ho T-H D, Mikkonen A: Expression of the 30 kd cysteine endoprotease b in germinating barley seeds. Cell biol int 17 (2): 205-212 (1993).

Marttila S, Jones B L, Mikkonen A: Differential localization of two acid proteases in germinating barley (Hordeum vulgare) seed. Physiologia Plantarum 93 (2): 317-327 (1995).

Mckee R A, Adams S, Matthews J A, Smith C J, Smith H: Molecular cloning of two cysteine proteases from paw-paw (carica-papaya). Biochem J 237 (1): 105-1101 (1986).

Minami A and Fukuda H: Transient and specific expression of a cysteine endopeptidase associated with autolysis during differentiation of Zinnia mesophyll cells into tracheary elements. Plant Cell Physiol 36 (8): 1599-1606 (1995).

Mino M, Inoue M: Hybrid vigor in relation to lipid and protein metabolism in germinating maize kernels. Jpn J Breed 38 (4): 428-436 (1988).

Nong V H, Becker C, Muentz K: cDNA cloning for a putative cysteine protease from developing seeds of soybean. Biochim et Biophys Acta 1261 (3): 435-438 (1995).

Okayama H, Kawaichi M, Brownstein M, Lee F, Yokota T, Arai K: High-efficiency cloning of full-length cDNA: construction and screening of cDNA expression libraries from mammalian cells. Meth. in Enzymology 154: 3-27 (1987).

Pautot V, Brzezinski R, Tepfer M: Expression of a mouse metallothionein gene in transgenic plant tissues. Gene 77:133-140 (1989).

Pladys D, Vance C P: Proteolysis during development and senescence of effective and plant gene-controlled ineffective alfalfa nodules. Plant Physiol 103 (2): 379-384 (1993).

Praekelt U M, Mckee R A, Smith H: Molecular analysis of actinidin the cysteine protease of Actinidia chinensis. Plant Mol Biol 10 (3): 193-202 (1988).

Revell d f, Cummings N J, Baker K C, Collins M E, Taylor M A J, Sumner I G, Pickersgill R W, Connerton I F, Goodenough P W: Nucleotide sequences and expression in Escherichia coli of cDNA encoding papaya protease omega from Carica papaya. Gene 127 (2): 221-225 (1993).

Sambrook J, Fritsch E F, Maniatis T: Molecular cloning, a laboratory manual. Cold Spring Harbor Laboratory, Cold Spring Harbor, N.Y. (1989).

Sanger F, Milkin S, Coulson A R: DNA sequencing with chain-terminating inhibitors. Proc Natl Acad Sci USA 74: 5463-5467 (1977).

Schaffer M A, Fischer R L: Analysis of mRNAs that accumulate in response to low temperatures identifies a thiol protease gene in tomato. Plant Physiol 87: 431-436 (1988).

Shimada T,Hiraiwa N, Nishimura M, Hara-Nishimura I: Vacuolar processing enzyme of soybean that converts proproteins to the corresponding mature forms. Plant Cell Physiol 35 (4): 713-718 (1994).

Shintani A, Yamauchi D, Minamikawa T: Nucleotide sequence of cDNA for a putative cysteine protease from rice seeds. Plant Physiol 107 (3): 1025 (1995).

Shutov A D, Vaintraub I A: Degradation of storage proteins in germinating seeds. Phytochemistry 26 (6): 1557-1566 (1987).

Smart C M, Hosken S E, Thomas H, Greaves J A, Blair B G, Schuch W: The timing of maize leaf senescence and characterisation of senescence-related cDNAs. Physiol Plant

93 (4): 673-682 (1995).

Takeda O, Miura Y, Mitta M, Matsushita H, Kato I, Abe V, Yokosawa H Ishii S-I: Isolation and analysis of cDNA encoding a precursor of Canavalia ensiformis asparaginyl endopeptidase (Legumain). Journal of Biochemistry 16 (3): 541-546 (1994).

Taylor M A J, Baker K C, Briggs G S, Connerton I F, Cummings N J, Pratt K A, Revell D F, Freedman R B, Goodenough P W: Recombinant pro-regions from papain and papaya protease IV are selective high affinity inhibitors of the mature papaya enzymes. Protein Engineering 8 (1): 59-62 (1995).

Terras R G, Torrekens S, Van Leuven F, Osborn R W, Vanderleyden J, Cammue B P A, Broekaert W F: A new family of basic cysteine-rich plant antifungal proteins from Brassicaceae species. FEBS Lett 316 (3): 233-240 (1993).

Thomas M P, Topham C M, Kowlessur D, Mellor G W, Thomas E W, Whitford D, Brocklehurst K: Structure of chymopapain M the late-eluted chymopapain deduced by comparative modelling techniques and active-centre characteristics determined by pH-dependent kinetics of catalysis and reactions with time-dependent inhibitors: The Cys-25-His-159 ion-pair is insufficient for catalytic competence in both chymopapain M and papain. Biochemical Journal 300 (3): 805-820 (1994).

Valpuesta V, Lange N E, Guerrero C, Reid M S: Up-regulation of a cysteine protease accompanies the ethylene-insensitive senescence of daylily (Hemerocallis) flowers. Plant Mol Biol 28 (3): 575-582 (1995).

Vernet T, Berti P J, De Montigny C, Musil R, Tessier D C, Menard R, Magny M-C, Storer A C, Thomas D Y: Processing of the papain precursor: the ionization state of a conserved amino acid motif within the Pro region participates in the regulation of intramolecular processing. J Biol Chem 270 (18): 10838-10846 (1995).

Volkel H, Kurz U, Linder J, Klumpp S, Gnau V, Jung G and Schultz J E: Cathepsin L is an intracellular protease in Paramecium tetraurelia. Purification, cloning, sequencing and specific inhibition of its expressed propeptide. Eur J Biochem 238: 198-206 (1996).

Watanabe H, Abe K, Emori Y, Hosoyama H, Arai S: Molecular cloning and gibberellin-induced expression of multiple cysteine proteases of rice seeds (oryzains). J Biol Chem 266 (25): 16897-16902 (1991).

Wiederanders B, Broemme D, Kirschke H, Von Figura K, Schmidt B, Peters C: Phylogenetic conservation of cysteine proteases cloning and expression of a cDNA coding for human cathepsins. J Biol Chem 267 (19): 13708-13713 (1992).

Yamauchi D, Akasofu H, Minamikawa T: Cysteine endopeptidase from Vigna mungo: gene structure and expression. Plant Cell Physiol. 33 (6): 789-797 (1992).

1. An isolated DNA construct comprising:
a) a first DNA sequence comprising either an inducible promoter sequence responsive to the presence or absence of an exogenous inducer or a developmental gene promoter capable of initiating gene expression in a selected tissue or at a selected stage of development of an organism;
b) a second DNA sequence comprising a DNA sequence coding for a protease enzyme operably linked and under the control of the promoter sequence specified at (a);
whereby the presence or absence of the exogenous inducer or the activation of the developmental gene promoter specified at (a) results in expression of said protease enzyme.

2. A DNA construct comprising:
a) a first DNA sequence comprising either an inducible promoter sequence responsive to the presence or absence of an exogenous inducer or a developmental gene promoter capable of initiating gene expression in a selected tissue or at a selected stage of development of an organism;
b) a second DNA sequence comprising a DNA sequence coding for a protease enzyme capable of disrupting cell function operably linked and under the control of the promoter sequence specified at (a);
c) a third DNA sequence comprising a promoter sequence responsive to the presence or absence of an exogenous inducer or a developmental gene promoter capable of initiating gene expression in a selected tissue or at a selected stage of development of an organism; and
d) a fourth DNA sequence comprising a DNA sequence, the product of which is capable of inhibiting the protein specified in (b) operably linked and under the control of the promoter sequence specified at (c),
whereby the presence or absence of the exogenous inducer or the expression of the developmental gene promoter specified at (a) enables cell function to be disrupted and whereby the presence or absence of the exogenous inducer or the expression of the developmental gene promoter specified at (c) prevents or reduces the disruption of cell function.

3. A DNA construct according to claim 2 wherein the first DNA sequence further comprises an operator sequence responsive to a repressor protein coded for by a DNA sequence comprised within a fifth DNA sequence which is operably linked and under the control of a sixth DNA sequence comprising an inducible promoter sequence, responsive to the presence or absence of an exogenous inducer, whereby expression of the repressor protein prevents or reduces the disruption of cell function.

4. A DNA construct according to claim 2 or claim 3 wherein the fourth DNA sequence comprises a DNA sequence coding for a member of a family of proteins which are naturally associated with the DNA sequence specified in (b).

5. A DNA construct according to claim 4 wherein the fourth DNA sequence comprises a DNA sequence coding for a protease propeptide, a protease inhibitor or an antibody directed to the protein capable of disrupting cell function.

6. A DNA construct according to any one of claims 1 to 5 wherein the protease is a targeted or non-targeted cysteine protease enzyme.

7. A DNA construct according to any one of the preceding claims wherein the developmental gene promoter is an early seedling/seed promoter or a pathogen-induced promoter.

8. A DNA construct according to any one of the preceding claims capable of disrupting cell function in a plant cell or a mammalian cell.

9. Plant germplasm comprising a plant comprising a DNA construct of any one of claims 1 to 8.

10. A plant, plant seed or plant cell comprising a DNA construct of any one of claims 1 to 8.

11. A mammalian cell comprising a DNA construct of any one of claims 1 to 8.

12. The use of a DNA construct as defined in any one of claims 1 to 8 to disrupt cell function.

13. DNA construct comprising:
(a) a first DNA sequence comprising either an inducible promoter sequence responsive to the presence or absence of an exogenous inducer or a developmental gene promoter capable of initiating gene expression in a selected tissue or at a selected stage of development of a plant; and
(b) a second DNA sequence comprising a DNA sequence, the product of which is capable of inhibiting a protein capable of controlling senescence in a plant operably linked and under the control of the promoter sequence specified at (a),
whereby the presence or absence of the exogenous inducer or the activity of the developmental gene promoter enables the synthesis of the protein capable of controlling senescence to be down-regulated or its activity to be substantially reduced, thereby enabling the process of senescence to be slowed down.

14. A DNA construct according to claim 13 wherein the second DNA sequence comprises a DNA sequence coding for a member of a family of proteins which are naturally associated with the protein capable of controlling senescence.

15. A DNA construct according to claim 13 or claim 14 wherein the second DNA sequence comprises a DNA sequence coding for a protease propeptide or a partial sense or antisense RNA.

16. A DNA construct according to any one of claims 13 to 17 wherein the protein capable of controlling senescence is a protease enzyme.

17. A DNA construct according to claim 15 or claim 16 wherein the protease is a targeted or non-targeted cysteine protease.

18. A DNA construct according to any one of claims 13 to 17 wherein the developmental gene promoter is a senescence-induced promoter.

19. A DNA construct according to any one of claims 13 to 18 capable of down-regulating or substantially reducing the activity of the protein capable of controlling senescence in a plant cell.

20. Plant germplasm comprising a plant comprising a DNA construct of any one of claims 1 to 19.

21. A plant, plant seed or plant cell comprising a DNA construct of any one of claims 1 to 19.

22. The use of a DNA construct as defined in any one of claims 13 to 19 to down-regulate the synthesis or substantially reduce the activity of a protein capable of controlling senescence.

23. A DNA construct comprising:
(a) a first DNA sequence comprising either an inducible promoter sequence responsive to the presence or absence of an exogenous inducer or a developmental gene promoter capable of initiating gene expression in a selected tissue or at a selected stage of development of a plant; and
(b) second DNA sequence comprising a DNA sequence, the product of which is capable of down-regulating the synthesis or inhibiting a protein capable of controlling metabolism of stored protein in a plant operably linked and under the control of the promoter sequence specified at (a),
whereby the presence or absence of the exogenous inducer or the expression of the developmental gene promoter specified at (a) enables the synthesis of the protein capable of controlling metabolism of stored protein to be down-regulated or its activity to be substantially reduced, thereby altering the nature, the mobilisation and/or the distribution of stored products in plants.

24. A DNA construct according to claim 23 wherein the first DNA sequence further comprises an operator sequence responsive to a repressor protein coded for by a DNA sequence comprised within a third DNA sequence which is operably linked and under the control of an inducible promoter sequence, responsive to the presence of an exogenous inducer, coded for by a DNA sequence comprised within a fourth DNA sequence whereby expression of the repressor protein prevents alterations in the metabolism of stored proteins.

25. A DNA construct according to claim 23 or claim 24 further comprising a fifth DNA sequence comprising a DNA sequence coding for a heterologous protein which is operably linked and under the control of a sixth DNA sequence which comprises an inducible promoter, responsive to the presence or absence of an exogenous inducer, whereby expression of the heterologous protein gene prevents alterations in the metabolism of stored proteins.

26. A DNA construct according to any one of claims 23 to 25 wherein the protein capable of modifying the metabolism of stored protein in a plant is a protease enzyme.

27. A DNA construct according to claim 26 wherein the protein capable of modifying the metabolism of stored protein is a targeted or non-targeted cysteine protease enzyme.

28. A DNA construct according to any one of claims 23 to 27 wherein the second DNA sequence comprises a DNA sequence coding for a product which is naturally associated with the protein capable of modifying the metabolism of stored protein.

29. A DNA construct according to any one of claims 23 to 28 wherein the second DNA sequence comprises a DNA sequence coding for a protease propeptide.

30. A DNA construct according to any one of claims 23 to 29 wherein the second DNA sequence comprises a DNA sequence coding for a protease sense RNA or partial sense RNA.

31. A DNA construct according to any one of claims 23 to 29 wherein the second DNA sequence comprises a DNA sequence coding for a protease antisense RNA.

32. A DNA construct according to claim 30 or claim 31 wherein the second DNA sequence comprises a DNA sequence coding for a cysteine protease sense/partial sense or antisense RNA.

33. A DNA construct according to any one of claims 23 to 32 wherein the heterologous protein is a protease.

34. A DNA construct according to any one of claims 23 to 33 wherein the developmental gene promoter is an early seedling promoter.

35. A DNA construct according to any one of claims 23 to 34 capable of down-regulating the synthesis or substantially reducing the activity of the protein capable of modifying the metabolism of stored protein in plants.

36. A plant, plant seed or plant cell comprising a DNA construct of any one of claims 23 to 35

37. The use of a DNA construct as defined in any one of claims 23 to 35 to down-regulate the synthesis or substantially reduce the activity of a protein capable of modifying the metabolism of stored protein.
